SEQUENCE LISTING (48 sequences)

SEQ ID NO: 1 - DNA, 1410 bp, Aeromonas hydrophila
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatgcgcaagagctctccgggcttggcttcaacaacgtcagcctcagcgtgacccctgctgccaatcaa

SEQ ID NO: 2 - protein, 470 aa, Aeromonas hydrophila
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 3 - DNA, 1410 bp, Artificial Sequence
  Proaerolysin with a PSA sequence substituted for the furin site.
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccattcctccaagctgcagagtgtggacggcgctggtcaaggcctgaggctggagatcccgctcgatgcgcaagagctctccgggcttggcttcaacaacgtcagcctcagcgtgacccctgctgccaatcaa

SEQ ID NO: 4 - protein, 470 aa, Artificial Sequence
  Proaerolysin with a PSA sequence substituted for the furin site.
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQ

SEQ ID NO: 5 - protein, 6 aa, Homo sapiens
HSSKLQ

SEQ ID NO: 6 - DNA, 1410 bp, Artificial Sequence
  Proaerolysin with a PSA sequence substituted for the furin site.
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagccattcctccaagctgcagagtgccgacggcgctggtcaaggcctgaggctggagatcccgctcgatgcgcaagagctctccgggcttggcttcaacaacgtcagcctcagcgtgacccctgctgccaatcaa

SEQ ID NO: 7 - protein, 470 aa, Artificial Sequence
  Proaerolysin with a PSA sequence substituted for the furin site.
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSADGAGQGLRLEIPLDAQELSGLGFNNVSLSVTPAANQ

SEQ ID NO: 8 - protein, 8 aa, Artificial Sequence
  PSA cleavage site
HSSKLQSA

SEQ ID NO: 9 - DNA, 1410 bp, Artificial Sequence
  Proaerolysin with a PSA sequence substituted for the furin site.
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgactcccagttctatagcagcaatagtgtggacggcgctggtcaaggcctgaggctggagatcccgctcgatgcgcaagagctctccgggcttggcttcaacaacgtcagcctcagcgtgacccctgctgccaatcaa

SEQ ID NO: 10 - protein, 470 aa, Artificial Sequence
  Proaerolysin with a PSA sequence substituted for the furin site.
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSQFYSSNSVDGAGQGLRLEIPLDAQELSGLGFNNVSLSVTPAANQ

SEQ ID NO: 11 - protein, 6 aa, Artificial Sequence
  PSA cleavage site
QFYSSN

SEQ ID NO: 12 - DNA, 1410 bp, Artificial Sequence
  Proaerolysin with a PSA sequence substituted for the furin site.
gcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacggtataagtagtttccagagtagtgtggacggcgctggtcaaggcctgaggctggagatcccgctcgatgcgcaagagctctccgggcttggcttcaacaacgtcagcctcagcgtgacccctgctgccaatcaa

SEQ ID NO: 13 - protein, 470 aa, Artificial Sequence
  Proaerolysin with a PSA sequence substituted for the furin site.
AEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADGISSFQSSVDGAGQGLRLEIPLDAQELSGLGFNNVSLSVTPAANQ

SEQ ID NO: 14 - protein, 7 aa, Artificial Sequence
  PSA cleavage site
GISSFQS

SEQ ID NO: 15 - protein, 7 aa, Homo sapiens
KGISSQY

SEQ ID NO: 16 - protein, 7 aa, Homo sapiens
SRKSQQY

SEQ ID NO: 17 - protein, 7 aa, Homo sapiens
ATKSKQH

SEQ ID NO: 18 - protein, 7 aa, Homo sapiens
KGLSSQC

SEQ ID NO: 19 - protein, 7 aa, Homo sapiens
LGGSSQL

SEQ ID NO: 20 - protein, 7 aa, Homo sapiens
EHSSKLQ

SEQ ID NO: 21 - protein, 4 aa, Homo sapiens
SKLQ

SEQ ID NO: 22 - protein, 10 aa, Artificial sequence
  LHRH variant sequence
QHWSYGLRPG

SEQ ID NO: 23 - protein, 10 aa, Artificial Sequence
  LHRH variant sequence
EHWSYKLRPG

SEQ ID NO: 24 - protein, 397 aa, Artificial Sequence
  Variant proaerolysin peptide
EHWSYKLRPGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQ

SEQ ID NO: 25 - protein, 397 aa, Artificial Sequence
  Variant proaerolysin peptide
EHWSYKLRPGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDAQELSGLGFNNVSLSVTPAANQ

SEQ ID NO: 26 - protein, 476 aa, Artificial Sequence
  Proaerolysin with PSA sequence substituted for the furin site and an N-terminal His tag.
HHHHHHAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQ

SEQ ID NO: 27 - protein, 585 aa, Homo sapiens
  MISC_FEATURE (1)..(585): Human Albumin mature sequence
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMRADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 28 - protein, 4 aa, Artificial Sequence
  HK2-specific cleavage site
QKRR

SEQ ID NO: 29 - protein, 4 aa, Artificial Sequence
  HK2-specific cleavage site
KSRR

SEQ ID NO: 30 - protein, 4 aa, Artificial Sequence
  HK2-specific cleavage site
AKRR

SEQ ID NO: 31 - protein, 4 aa, Artificial Sequence
  HK2-specific cleavage site
KKRR

SEQ ID NO: 32 - protein, 4 aa, Artificial Sequence
  HK2-specific cleavage site
HKRR

SEQ ID NO: 33 - protein, 4 aa, Artificial Sequence
  HK2-specific cleavage site
KAFR

SEQ ID NO: 34 - protein, 4 aa, Artificial Sequence
  HK2-specific cleavage site
KAQR

SEQ ID NO: 35 - protein, 4 aa, Artificial Sequence
  HK2-specific cleavage site
KAKR

SEQ ID NO: 36 - protein, 4 aa, Artificial Sequence
  HK2-specific cleavage site
KARK

SEQ ID NO: 37 - protein, 4 aa, Artificial Sequence
  HK2-specific cleavage site
KAHR

SEQ ID NO: 38 - protein, 6 aa, Artificial Sequence
  HK2-specific cleavage site
HAQKRR

SEQ ID NO: 39 - protein, 6 aa, Artificial Sequence
  HK2-specific cleavage site
GGKSRR

SEQ ID NO: 40 - protein, 6 aa, Artificial Sequence
  HK2-specific cleavage site
HEQKRR

SEQ ID NO: 41 - protein, 6 aa, Artificial Sequence
  HK2-specific cleavage site
HEAKRR

SEQ ID NO: 42 - protein, 6 aa, Artificial Sequence
  HK2-specific cleavage site
GGQKRR

SEQ ID NO: 43 - protein, 6 aa, Artificial Sequence
  HK2-specific cleavage site
HEQKRR

SEQ ID NO: 44 - protein, 6 aa, Artificial Sequence
  HK2-specific cleavage site
GGAKRR

SEQ ID NO: 45 - protein, 6 aa, Artificial Sequence
  HK2-specific cleavage site
HEQKRR

SEQ ID NO: 46 - protein, 6 aa, Artificial Sequence
  HK2-specific cleavage site
GGKKRR

SEQ ID NO: 47 - protein, 6 aa, Artificial Sequence
  HK2-specific cleavage site
GGHKRR

SEQ ID NO: 48 - protein, 1061 aa, Artificial Sequence
  HSA fused to PRC302 with one PSA cleavage site linker
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMRADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLHSSKLQAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSHSSKLQSVDGAGQGLRLEIPLDAQELSGLGFNNVSLSVTPAANQ

(19) United States
(12) Patent Application Publication
(10) Pub. No.: US 2018/0148480 A1
(43) Pub. Date: May 31, 2018

(54) SYNTHETIC ENHANCEMENT OF THE T-CELL ARMAMENTARIUM AS AN ANTI-CANCER THERAPY

(71) Applicant: THE JOHNS HOPKINS UNIVERSITY, Baltimore, MD (US)

(72) Inventors: John T. Isaacs, Phoenix, MD (US); Samuel R. Denmeade, Ellicott City, MD (US); Nathaniel E. Brennen, Baltimore, MD (US)

(21) Appl. No.: 15/544,156

(22) PCT Filed: Jan. 15, 2016
(86) PCT No.: PCT/US2016/013568

§ 371 Date: Jul. 17, 2017

Related U.S. Application Data
(60) Provisional application No. 62/104,368, filed on Jan. 16, 2015.

(51) Int. Cl. C07K 14/195; C07K 14/725; C07K 14/705; C07K 16/30; C12N 5/0783

(52) U.S. Cl. CPC C07K 14/195; C07K 14/7051; C07K 14/70521; A61K 2039/5158; C12N 5/0636; C07K 2317/622; C07K 2319/03; C07K 16/30

(57) ABSTRACT

The present invention relates to the field of cancer. More specifically, the present invention provides compositions and methods for using synthetically enhanced T-cells to treat cancer. The present invention also provides a T-cell engineered (a) to express at least one CAR that binds tumor antigens; and (b) to inducibly express a prostate-specific antigen (PSA)-activated pro-aerolysin (PA) upon tumor antigen recognition by CAR.

### CROSS-REFERENCE TO RELATED APPLICATIONS

This application claims the benefit of U.S. Provisional Application No. 62/104,368, filed Jan. 16, 2015, which is incorporated herein by reference in its entirety.

### FIELD OF THE INVENTION

The present invention relates to the field of cancer. More specifically, the present invention provides compositions and methods for using synthetically enhanced T-cells to treat cancer.

### INCORPORATION-BY-REFERENCE OF MATERIAL SUBMITTED ELECTRONICALLY

This application contains a sequence listing. It has been submitted electronically via EFS-Web as an ASCII text file entitled “P12863-02_ST25.txt.” The sequence listing is 61,974 bytes in size, and was created on Jan. 14, 2016. It is hereby incorporated by reference in its entirety.

### BACKGROUND OF THE INVENTION

Prostate cancer (PCa) represents the largest number of new cancer diagnoses in men each year (1). Despite recently approved therapies, such as abiraterone and sipuleucel-T, more than 30,000 men will succumb to cancer-related morbidities associated with PCa metastasis this year in the United States alone (2). Consequently, innovative therapeutic strategies capable of treating metastatic disease are desperately needed to improve long-term patient survival.

It has become clear that tumors are more than a collection of malignant cells harboring genetic and epigenetic changes that allow them to bypass normal physiological controls on cell growth. Tumors are actually composed of a complex network of multiple cell types, including endothelial cells, macrophages, pericytes, fibroblasts, and leukocytes; all of which have been shown to play critical roles in carcinogenesis and must be subverted for a carcinoma to ultimately progress to a metastatic phenotype (3-4). While a deeper understanding of the pathophysiological role of each of these cell types in cancer will undoubtedly elucidate novel targets, their presence within the tumor microenvironment raises the exciting possibility of exploiting their tumor trafficking properties to deliver chemotherapeutic agents. Cell-based therapeutic platforms have been called ‘the next generation of medicine’ and represent a growing area of intense research in oncology and other diseases (5). The promise of these cell-based therapies is derived from harnessing the power of evolution and utilizing the intrinsic properties within these cells for therapeutic benefit.

### SUMMARY OF THE INVENTION

T-cells can be armed with a highly potent cytotoxic agent capable of killing cancer cells independent of these immunosuppressive signals and used as a cell-based delivery vector by exploiting their innate tumor tropism. PSA-activated proaerolysin (PA) is a recombinant bacterial protoxin that rapidly kills cells in a proliferation-independent manner at low nanomolar (nM) concentrations by forming pores in the plasma membrane following PSA-dependent cleavage of the inhibitory domain. In essence, the T-cells would serve as a “Trojan Horse” to selectively deliver the protoxin to sites of advanced PCa. The T-cells are genetically-engineered such that the protoxin is only expressed and secreted following T-cell recognition of PSMA-positive cells through a chimeric antigen receptor (CAR) expressed on the T-cell surface. CARs are synthetic T-cell receptors (TcRs) that can be engineered to recognize a tumor- or tissue-specific antigen, such as PSMA. Not only will T-cell potency be enhanced through protoxin secretion, but greater specificity can be achieved using this combinatorial antigen recognition (PSMA) and activation (PSA) strategy to limit toxicity to non-target tissues. Importantly, enzymatically-active PSA is only present in the prostate and at sites of PCa, including metastases, because circulating PSA is bound to ubiquitous protease inhibitors. The enhanced potency and specificity of the proposed strategy has the potential to significantly alter the clinical application of immunotherapies in the future and drive dramatic therapeutic responses in patients with metastatic PCa.

Accordingly, in one aspect, the present invention provides engineered T-cells. In certain embodiments, a T-cell is engineered to express a prostate-specific antigen (PSA)-activated pro-aerolysin (PA) upon tumor antigen recognition by a chimeric antigen receptor (CAR) expressed on the surface of the T-cell. In a specific embodiment, the T-cell expresses more than one type of tumor antigen recognizing CAR. In particular embodiments, the tumor antigen comprises prostate specific membrane antigen (PSMA) and/or prostate stem cell antigen (PSCA).

The present invention also provides a T-cell engineered (a) to express at least one CAR that binds tumor antigens; and (b) to inducibly express a prostate-specific antigen (PSA)-activated pro-aerolysin (PA) upon tumor antigen recognition by CAR. In other embodiments, the present invention provides a T-cell engineered to express a protoxin upon tumor antigen recognition by a CAR expressed on the surface of the T-cell. In a specific embodiment, the protoxin is activated via cleavage by a cancer specific protease. Tumor antigens that can be used in the compositions and methods of the present invention include, but are not limited to, α-Folate receptor, CAIX, CAIX, CD19, CD19, CD19, CD19, CD19, CD19, CD19, CD19, CD19, CD19, CD19, CD19, CD19, CD19, CD19, CD19, CD19, CD20, CD20, CD20, CD20, CD20, CD20, CD20, CD22, CD30, CD30, CD33, CD33, CD44v7/8, CEA, CEA, CEA, CEA, CEA, CEA, EGP-2, EGP-2, EGP-40, erb-B2, erb-B2, erb-B2, erb-B2, erb-B2, erb-B 2,3,4, erb-B 2,3,4, FBP, FBP, Fetal acetylcholine receptor, GD2, GD2, GD2, GD2, GD2, GD3, GD3, Her2/neu, Her2/neu, Her2/neu, Her2/neu, IL-13R-a2, IL-13R-a2, IL-13R-a2, KDR, k-light chain, k-light chain, LeY, LeY, L1 cell adhesion molecule, MAGE-A1, MAGE-A1, Mesothelin, Mesothelin, Mesothelin, Murine CMV infected cells, MUC1, NKG2D ligands, Oncofetal antigen (h5T4), PSCA, PSMA, PSMA, PSMA, TAA targeted by mAb IgE, TAG-72, and VEGF-R2.

### BRIEF DESCRIPTION OF THE FIGURES

FIG. 1. Graphical illustration of Chimeric Antigen Receptors (CARs).

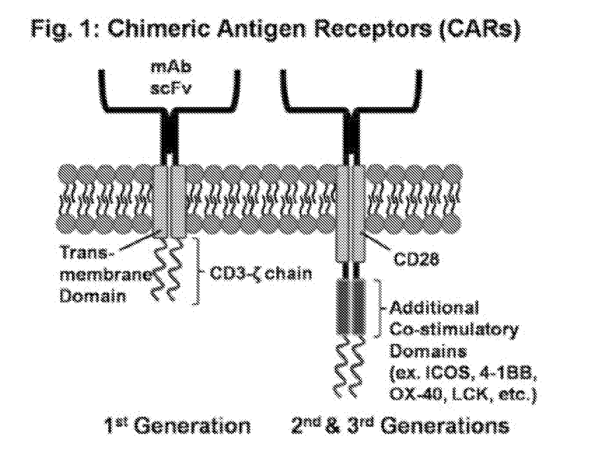

FIG. 2. Generation of PSA-activated proaerolysin.

FIG. 3. Mammalian expression and secretion of PSA-activated proaerolysin.

FIG. 4. Analysis of T-cells in a primary prostatectomy specimen by (A) flow cytometry and (B) IHC.

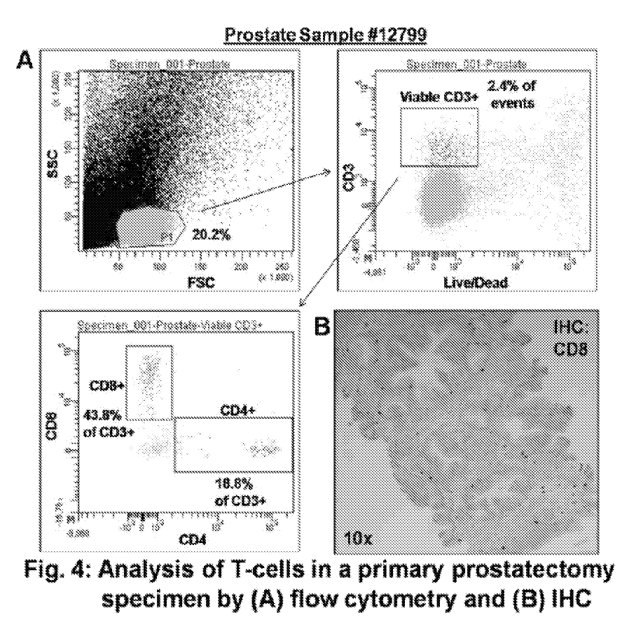

FIG. 5. Combinatorial strategy for enhanced specificity and therapeutic efficacy of CAR-expressing T-cells. Expression and secretion of PSA-activated proaerolysin (PA) is dependent on antigen recognition (PSMA) by genetically-engineered T-cells to induce a downstream signaling cascade within the T-cell that leads to activation of a promoter (e.g., an IFN-γ). Protoxin activation following secretion into the tumor microenvironment is dependent on the presence of enzymatically-active PSA, which is only found in the prostate and PCa primary and metastatic tumors. PSA-dependent cleavage of the inhibitory domain leads to aerolysin oligomerization and pore formation, which results in rapid cell lysis at picomolar concentrations following membrane insertion.

FIG. 6. Targeted insertion of PSA-activated proaerolysin into the PIG-A locus using Zinc-finger nucleases (ZFNs).

FIG. 7. Combinatorial antigen recognition and protoxin activation for enhanced specificity.

FIG. 8. Sensitive detection of PSA-activated proaerolysin by (A) western blot and (B) sandwich ELISA.

FIG. 9. PSA-dependent activation and lysis of RBCs by PSA-activated Proaerolysin (PA).

FIG. 10. PSA-activated proaerolysin with a mutation in the GPI-anchor binding domain (R624A) have reduced toxicity against LNCaP prostate cancer cells.

FIG. 11. PC3 cells expressing PSMA or the vector control.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Pro-Aerolysin as a ‘Molecular Grenade’

Pro-aerolysin (PA) is produced and secreted by the aquatic Gram-negative bacteria Aeromonas hydrophilia as a 52 kD water-soluble dimer (51-52). Mechanistically, PA binds to glycophosphatidylinositol (GPI)-anchored proteins (GPI-APs) present on the surface of all mammalian cells, where it undergoes proteolytic activation by furin-like proteases in the extracellular fluid (52). Cleavage of this C-terminal inhibitory domain induces oligomerization and membrane insertion by exposing specific hydrophobic domains that permit heptameric pore formation. The resulting pore leads to rapid cell death at very low picomolar concentrations by disrupting the plasma membrane (51-53). Pore-forming toxins are particularly well-suited for use as cytotoxic agents in PCa therapy because they are able to potently kill cells in a proliferation-independent manner (53). This is critical because PCa typically has a proliferative fraction <5%, which makes it resistant to traditional cell cycle-dependent chemotherapy (54). Intrinsic in this mechanism of action is the fact that PA is non-selective and extremely toxic to all cell types in its native form. We have previously demonstrated that wild-type PA is toxic to both human PCa cell lines (LNCaP, PC3, DU145, LAPC-4, and CWR22Rv1) and non-PCa cells [TSU (bladder), SN12C (renal), and TT (thyroid)] at LD50 concentrations ≤50 pM (53). In vivo, a single intravenous dose of only 0.1 μg kills 100% of animals within 24 hrs (LD100)(53). In fact, based on PA's potency, as little as one pore per cell has been calculated to result in cell death, making PA an extremely toxic, cell-type independent agent. Furthermore, due to PA's mechanism of action (i.e., pore formation), selective outgrowth of resistant subclones within the tumor is unlikely. In summary, PA is ideal for the proposed ‘molecular grenade’ strategy because it potently kills tumor cells independent of target expression, cellular internalization, and cell cycle progression; however, its lack of specificity necessitates the use of a protoxin strategy to selectively target its lytic potential to PCa and spare toxicity to normal tissues.

Generation of PSA-Activated Proaerolysin.

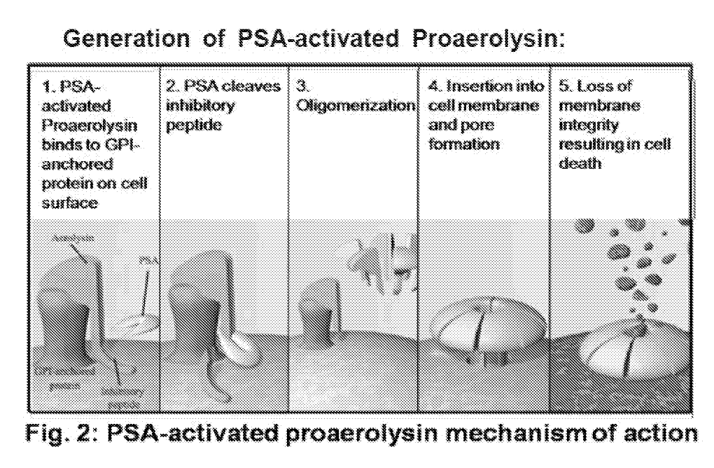

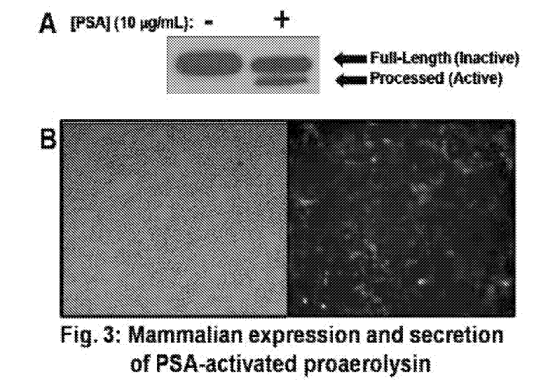

To overcome this lack of specificity, we have previously generated a prostate-specific antigen (PSA)-activated recombinant form of PA (FIG. 2)(53). PSA is a chymotrypsin-like protease only expressed by normal and malignant prostate epithelial cells. Furthermore, enzymatically-active PSA is secreted at high levels into the extracellular fluid by PCa cells at sites of metastasis (55). Importantly, binding to serum protease inhibitors, such as α1-antichymotrypsin and α2-macroglobulin, inactivates PSA upon entering circulation (55). Generation of this PSA-activated protoxin from wildtype PA was accomplished using site-directed mutagenesis to replace the native activation domain with a PSA-specific cleavage sequence, HSSKLQ (56). The mutated gene was then subcloned into the pMMB66HE vector for amplification in E. coli, and a His-tag was fused onto the C-terminus to aid in purification. This location was selected to ensure that only full-length, non-activated PA was isolated using affinity purification. PSA-dependent cleavage of the inhibitory domain has been confirmed (FIG. 3A). Importantly, intraprostatic injections into the PSA-producing monkey prostate produced no toxicity in periprostatic tissues, including the lateral pelvic fascia, anal sphincter, urethra, urinary bladder, rectum or other distant organs; thereby, demonstrating that the toxin does not re-enter systemic circulation once activated (53). Additionally, PSA-activated PA has been administered to >130 patients and is entering phase III registration trials as a local therapy for symptomatic BPH (57). While highly effective as a local therapy, its therapeutic index as a systemic agent is limited as result of binding to ubiquitous GPI-APs present on cells throughout the body. Consequently, a “Trojan Horse” strategy for protoxin delivery is needed for systemic applications.

To accomplish this goal, the PSA-activated PA transgene has been subcloned into a pLVX-AcGFP1-N1 lentiviral vector for mammalian expression. The vector has been modified to include a T2A ‘self-cleaving’ peptide sequence between the transgene and GFP to ensure stoichiometric expression of both proteins. The PSA-activated PA-T2A-GFP sequence is expressed as a single transcript, which is post-transcriptionally separated by an endogenous ‘ribosome skipping’ mechanism (58). Mammalian expression and secretion of PSA-activated PA has been confirmed (FIGS. 3A and B).

T-Cells as ‘Biological Microfactories’.

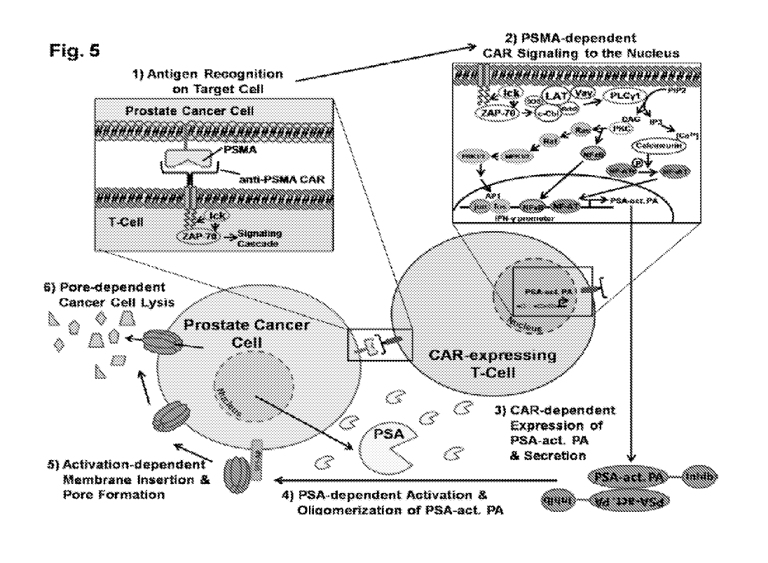

Delivery of therapeutic amounts of the protoxin is dependent upon both the number of cells trafficking to the tumor and the amount of drug delivered per cell. The latter are enhanced using a genetic engineering strategy to generate T-cells that secrete the protoxin upon antigen recognition by a PSMA-targeted CAR. Prostate-specific membrane antigen (PSMA) is expressed on the surface of prostate epithelial cells and is upregulated in both primary and metastatic cancer lesions (64-68). Additionally, PSMA expression has been detected on the tumor neovasculature, but not on normal endothelial cells, in multiple tumor types (39,69-72). Thus, PSMA represents a good tumor-associated antigen for CAR targeting (32,39,73-75); however, PSMA is also expressed in non-prostatic tissue, including the brain and proximal tubules of the kidney (39,76-77). Thus, a combinatorial strategy involving a second regulatory step, such as PSA-mediated protoxin activation, is needed to prevent potential ‘on-target, off-tumor’ effects (FIG. 5).

Retroviral vectors preferentially insert into non-oncogenic regions in T-cells; thereby, making them less susceptible to insertional oncogenesis (78-80). Furthermore, numerous clinical trials using retrovirally-transduced T-cells have been performed over the past decade with no reports of retroviral transformation. Despite this safety, there is a rational reason to utilize targeted genomic insertion of the protoxin. As discussed, PA binds to GPI-APs on the surface of cells, which helps facilitate pore formation and membrane insertion. GPI-anchors are a post-translational modification synthesized in a complex series of reactions involving more than 20 different gene products (81). The first of these biosynthesis steps is catalyzed by an enzyme encoded for by the phosphatidylinositol glycan anchor biosynthesis, class A (PIG-A) gene (81).

Patients with mutations in this gene have a rare disease known as paroxysmal nocturnal hemoglobinuria (PNH) characterized, in part, by hemolytic anemia resulting from defects in the complement cascade (82). Blood cells in these patients are resistant to aerolysin-induced hemolysis because they lack GPI-APs, or aerolysin ‘receptors’, on their surface (83). An observation that has led to the development of a clinical diagnostic assay for PNH (83). Therefore, targeted disruption of the PIG-A gene would render the genetically engineered T-cells resistant to PA (83); thereby, preventing self-sterilization because the cells would be unable to bind the secreted protoxin. In essence, this would turn the protoxin-expressing T-cells into biological ‘microfactories’ capable of secreting large quantities of the protoxin into the tumor microenvironment upon CAR stimulation. Importantly, although PNH patients have severe pathophysiological manifestations, these are derived from defects in the hematopoietic system. Therefore, T-cells lacking GPI-anchors would have no pathological consequences when re-infused, because the patient retains their full repertoire of normal cells, including hematopoietic stem cells, with an intact GPI-anchor biosynthesis pathway. Critically, PNH patients do not have an increased infection rate or known problems with T-cell trafficking (84-86); therefore, knocking out GPI-anchor biosynthesis should have no negative effects on their ability to traffic to sites of cancer.

Genetic Engineering of T-Cells.

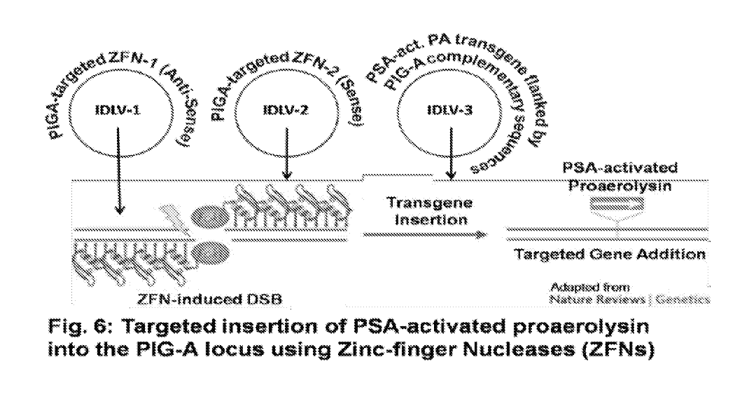

To accomplish this targeted insertion of the protoxin transgene into the PIG-A locus, zinc-finger nuclease (ZFN) technology is used. ZFNs are hybrid proteins generated by fusing the sequence-specific DNA-binding domain of zinc-finger proteins to the non-specific endonuclease domain of the Fok1 restriction enzyme (87-88). By using a pair of integration-deficient lentiviral vectors (IDLVs) encoding the nuclease fused to either the sense or antisense PIG-A sequence-specific DNA-binding domain, ZFNs bind and form an obligate heterodimer at the targeted locus to generate a double strand break. When this pair of IDLVZFNs is used in combination with a third IDLV containing a gene of interest flanked by complementary sequences to the insertion site, homologous recombination occurs. In combination with IDLVs, ZFNs have been used to introduce or edit genes of interest in mammalian cells, including T-cells (89), at predetermined chromosomal locations, including the PIG-A gene (FIG. 6)(90-93).

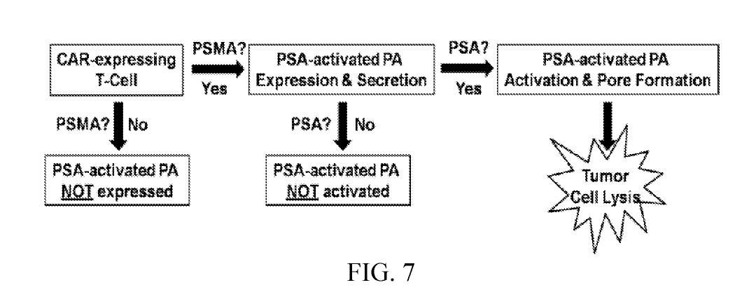

Research Strategy: The overall aim of this proposal is to genetically engineer T-cells to express and secrete a PSA-activated pore-forming protoxin into the PCa microenvironment upon chimeric antigen receptor (CAR) binding to a PSMA-positive cell. Enhanced specificity is achieved through combinatorial antigen recognition and protoxin activation (FIG. 7); thereby, limiting toxicity to normal host tissues as a result of T-cell trafficking to non-tumor tissue. Furthermore, generation of T-cells capable of producing large quantities of the protoxin will significantly increase the efficacy of current CART modalities by significantly decreasing the effector/target ratio.

To accomplish this proposal, proof-of-principle studies are performed to determine whether protoxin-expressing T-cells have a therapeutic advantage over antigen-specific cytotoxic T-cells (CTLs) alone using an immunocompetent transgenic model of autochthonous prostate cancer. Next, the strategy is adapted to a more clinically relevant scenario by genetically-engineering T-cells to express the PSMA-targeted CAR and a therapeutic amount of PSA-activated PA. Finally, the genetically engineered T-cells are evaluated using in vitro and in vivo preclinical models to determine the toxicity, specificity, and efficacy of the strategy. If successful, this strategy is then applied to other tumor types by engineering alternative tissue- or tumor-specific targets into the system.

Specific Aim 1: Engineer and evaluate a T-cell delivery vector armed with a protoxin in an immunocompetent mouse model of prostate cancer (PCa). The double transgenic ProTRAMP model (ProHA×TRAMP) develops autochthonous prostate cancers that express hemagglutinin (HA)(94). Despite the caveats associated with the TRAMP model, including its neuroendocrine phenotype, its practical use for the preclinical development of immunotherapies has been validated by the recent FDA-approval of ipilimumab, which was initially developed using this model (95). The ProTRAMP model has the added advantage of being able to utilize Clone 4 TCR transgenic animals to generate T-cells specific for a MHC Class I-restricted HA peptide (96) for antigen-specific CTL controls in proof-of-principle efficacy studies. Adoptive transfer of HA-specific CD8+ T-cells into tumor-bearing ProTRAMP mice generates tumor-specific T-cells with a nonfunctional phenotype due to the tolerogenic tumor microenvironment (94,97). Though an anti-tumor effect can be demonstrated in this model when therapy is initiated at early stages of disease with minimal tumor burden (at 10 wks with PIN-like lesions), more established tumors (>12 wks), such as those seen at clinical presentation, ultimately escape immunosurveillance and progress, killing the host (97). Though large tumors are not inherently resistant to therapy (37), the large tumor burden overwhelms the therapeutic response, and therefore, does not significantly improve overall survival in these patients (98). This implies that the lack of response is due to insufficient delivery and potency of the therapeutic agent, which in this case are CTLs. Therefore, this model represents an idealized system with a tissue-/tumor-specific antigen in which to test the ‘value added’ of administering protoxin-expressing T-cells over their cognate antigen-specific counterparts using a model that is refractory to current forms of immunotherapy (i.e., large established and widely disseminated tumors). Though mice do not express PSA, the wild-type PA construct can be placed under the control of a HA-targeted CAR for inducible expression of the protoxin within the prostate. Wildtype PA is activated by ubiquitous furin-like proteases (52); and therefore, would be rapidly activated following expression induced by HA recognition.

Sub-Aim 1a: Engineer naïve T-cells (TN) to express proaerolysin (PA) upon HA recognition by a CAR. An anti-HA CAR is generated by cloning the HA-scFv from the immunoglobulin genes of the HKPEG-1 hybridoma (ATCC #CCL-189), which produces an antibody specific to the Sa domain of HA, by PCR amplification of the variable regions of the heavy (VH) and light (VL) chains following cDNA synthesis using previously described methods and sequence-specific primers (99). Next, standard cloning techniques (100-101) are used to construct the following recombinant HA-scFv fusion protein: 5′-CD8 leader sequence-VH- (Gly-Ser2)5 linker-VL-CD8a hinge and transmembrane domains-CD3 chain-3′ (75); followed by cloning into a gammaretroviral SFG vector containing an internal ribosomal entry site (IRES)-GFP expression cassette (102). Viral particles are produced and purified following transient transfection of HEK293T according to previously published protocols (100). Peripheral blood mononuclear cells (PBMCs) are isolated using Ficoll gradients, and subsequently, depleted of B-cells and monocytes by incubation at 4° C. for 1 hr with Dynabeads targeting CD19 and CD14, respectively (Invitrogen). The remaining cells are assessed for CD3 enrichment by flow cytometry using an anti-mouse CD3 antibody (Clone 17A2, PE-conjugated, BD PharMingen). This fraction can be further enriched for naïve T-cells (TN) by positive selection for CD62L and negative selection for CD44 using antibody-conjugated magnetic beads (Miltenyi). The TN-enriched fraction are plated at a density of 2×106/mL and activated for 48 hrs with 2 μg/mL phytohemmaglutinin. Next, these cultures are transduced with viral particles containing the anti-HA CAR expression cassette twice by spinoculation for 1 hr before plating on retronectin-coated plates for 48 hrs in the presence of IL-2 (20 U/mL). Flow cyometry is used to determine transduction efficiency (GFP) and to sort a pure population for transduction with the IFNγ promoter-protoxin transgene described below.

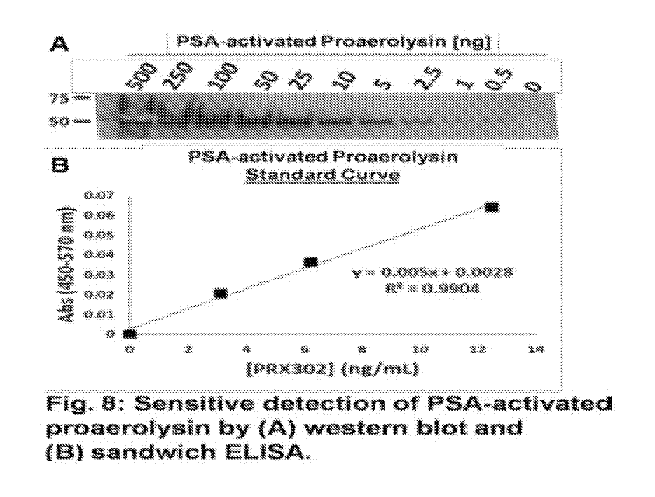

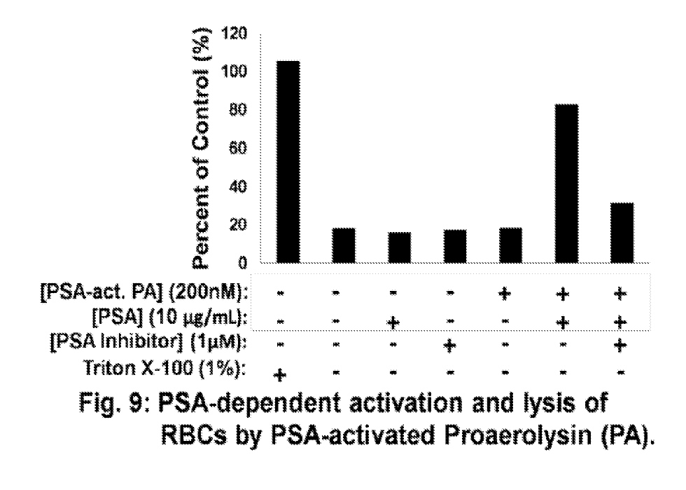

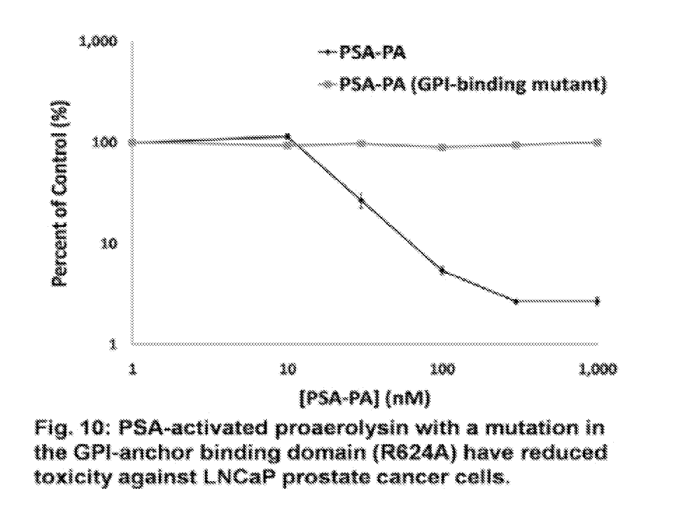

In particular embodiments, the IFN-γ promoter/enhancer (Addgene, Plasmid 17598) is cloned upstream of the wildtype PA transgene contained in the pMMB66HE vector (53). This IFNγ promoter-PA transgene is then subcloned into a previously generated donor vector containing a PGKHygroR gene flanked by homology arms to PIG-A (93). Importantly, this donor vector containing the PIGA homology domains, in addition to plasmids encoding the pair of previously described PIGA-targeted ZFNs (93) have already been obtained from the Cheng and Joung laboraties, respectively. Dr. Linzhao Cheng, a Professor in the Institute for Cell Engineering at Johns Hopkins, is an internationally recognized expert on the construction and use of lentiviral vectors and ZFNs (93,103-105). Dr Cheng has a history of collaboration with the present inventors on lentiviral-based projects to develop genetically modified MSCs (100). Following successful transduction with both expression vectors as determined by GFP expression and hygromycin resistance, these genetically-engineered T-cells are expanded on PC3 cells expressing the B7 co-stimulatory molecule (32) in the presence of IL-2 for use in subsequent studies. Protoxin expression following CAR stimulation by recombinant soluble HA (94) are evaluated using western blot and ELISA assays that have been developed within the lab (FIG. 8). Protoxin functionality is demonstrated using a standard hemolysis assay (FIG. 9) according to previously published methods (44,53) in the presence or absence of a boronic acid-based PSA inhibitor incorporating a bromopropylglycine group, Ahx-FSQn(boro)Bpg (Ki=72 nM) (106). Reduced sensitivity of the transduced T-cells to PA toxicity as a result of GPI-anchor loss due to PIG-A integration is demonstrated using standard MTT (3-[4,5-dimethylthiazol-2-yl]-2,5 diphenyl tetrazolium bromide)(FIG. 10) and clonogenic assays using previously published methods (39,43-44,53).

Sub-Aim 1b: Demonstrate superior efficacy of protoxin-expressing T-cells in an autochthonous model of PCa. ProTRAMP mice expressing HA driven by the prostate-specific minimal rat probasin promoter were developed by Dr. Charles Drake, and are available in the Johns Hopkins Animal Resources Facility (94). Clone-4 TCR transgenic mice (96) are also available in the JH Animal Resources Facility through this same collaboration and are used to obtain HA-specific CD8+ T-cells according to previously published protocols (97). Briefly, Clone-4 donor mice are sacrificed via CO2 asphyxiation prior to harvesting spleens and axillary lymph nodes. These tissues are homogenized and RBCs lysed using an RBC lysis solution (Miltenyi). Magnetically-labeled beads (Miltenyi) are used to purify CD8+ T-cells according to the manufacturer's protocol. After purification, cells are washed twice and resuspended in HBSS for injections.

The anti-tumor potency of the genetically-modified T-cells expressing HA-inducible PA generated in Sub-Aim 1b are compared to the HA-specific CD8+ T-cells isolated from Clone-4 donor mice in the ProTRAMP model. HA-specific or protoxin-expressing T-cells (1×106) are injected in 0.2 mL HBSS into the tail vein of 16 wk old ProTRAMP mice. TRAMP mice begin to develop histological evidence of PIN-like lesions around 10 wks of age and progress to a highly invasive phenotype with widely metastatic disease by 20 wks (107); therefore, these animals will have a substantial tumor burden that is refractory to standard ACT therapy, akin to what is frequently seen at clinical presentation. Mice are monitored for signs of distress and weight loss. All mice are sacrificed at 24 wks of age via CO2 asphyxiation, if not indicated earlier, and wet weights of the urogenital tract are obtained as a measure of disease burden. Ventral prostate lobes are isolated and bisected. Half are fixed, paraffin-embedded, and sectioned using a cryostat for histology to determine T-cell distribution and analysis of tumor grade in a blinded fashion as previously described (43,108). The remaining half are digested into a single cell suspension and processed by flow cytometry to quantify the number of infiltrating T-cells according to previously published protocols (47,108). Clinical data suggests that pre-conditioning the host with chemotherapy or total body irradiation (TBI) prior to adoptive cell transfer (ACT) increases engraftment and therapeutic efficacy (109-115). Therefore, animals are treated with either cyclophosphamide (250 mg/kg I.P.) to deplete Tregs or sublethal lymphodepleting TBI (5 Gy) using a Nordion Gammacell 40 small animal irradiator located in the Experimental Irradiator Core facility prior to T-cell infusion and compared to homing efficiency in unconditioned hosts.

If an insufficient therapeutic response is observed, various strategies can be employed to further boost tumor trafficking and/or protoxin expression. For example, multiple T-cell infusions at weekly intervals can be administered. Additionally, high-dose IL-2 in combination with antigenic stimulation, which is commonly used for T-cell expansion, has been shown to drive terminal differentiation to a TEFF phenotype (32,60). Evidence suggests that culturing T-cells in other γ-chain signaling (γc) cytokines, such as IL-15 (116) and IL-21 (117), or inhibition of metabolic and developmental pathways, such as PI3K (118), β-catenin (119), TGF-β (20), and Larginine metabolism (18); with small molecules may preserve a more naïve differentiation status, which are believed to have greater engraftment potential (60-61). T-cells can be cultured under these conditions to determine whether PCa homing can be further enhanced. Future studies analyzing chemokine receptor expression profiles on PCa-homing T-cells relative to those present in circulation and secondary lymphoid tissues may also help to elucidate a prostate-specific homing ‘address’ that can be used to further boost tumor trafficking (113-114,120-123). To enhance protoxin expression, the IFN-γ promoter can be modified to increase expression by incorporating additional enhancer elements or exchanged for a stronger promoter, such as TNFα. Additionally, transgene ‘stacking’, in which multiple copies of the protoxin are inserted into the PIG-A locus, can be used to further increase protoxin expression.

Specific Aim 2: Engineer and evaluate a T-cell protoxin delivery vector for human PCa. Aim 2 will generate genetically-engineered T-cells that express PSA-activated PA upon binding of an anti-PSMA CAR to target cells. Subsequently, the specificity, toxicity, and efficacy of these dual-targeted protoxin-expressing T-cells are evaluated in preclinical models of PCa.

Sub-Aim 2a: Genetically-engineer human naïve T-cells (TN) to express and secrete PSA-activated proaerolysin (PA) following PSMA-dependent CAR signaling. To construct the anti-PSMA CAR, a PSMAscFv are cloned from the immunoglobulin genes of the J591 hybridoma (ATCC #HB-12126) by PCR amplification of the variable regions of the heavy (VH) and light (VL) chains following cDNA synthesis using previously described methods and sequence-specific primers (99). Next, standard cloning techniques (100-101) are used to construct the following recombinant PSMA-scFv fusion protein: 5′-CD8 leader sequence-VH-(Gly-Ser2)5 linker-VL-CD8α hinge and transmembrane domains-CD3 ζ chain-3′ (75); followed by cloning into a gammaretroviral SFG vector containing an internal ribosomal entry site (IRES)-GFP expression cassette (102). Importantly, an anti-PSMA CAR of this composition has already been successfully generated (75); thereby, validating this methodology. Critically for the success of the proposed strategy, PSMA-specific activation of this first-generation construct was shown to stimulate intracellular signaling, but was not strong enough to drive full T-cell activation in the absence of co-stimulatory signals provided by the target cell (75). Because we are relying on CAR-dependent intracellular signaling to drive expression of the protoxin and want to prevent activation of endogenous cytolytic functions to prevent ‘off-tumor, on-target’ effects, this represents an ideal construct for use in this strategy (in contrast to its originally designed function). Viral particles are produced and purified following transient transfection of HEK293T according to previously published protocols (100). Human TN are isolated from PBMCs using a naïve pan T-cell isolation kit (Miltenyi) based on negative selection of non-target cells using magnetic beads and a labeling cocktail containing antibodies against HLA-DR, CD14, CD15, CD16, CD19, CD25, CD36, CD56, CD57, CD45RO, CD123, CD235a, CD244, and TCR γ/δ. The purity of this TN-enriched fraction are assessed by flow cytometry and used for transduction as described above. Flow cytometry is also used to determine transduction efficiency (GFP) and sort for a pure population for subsequent transduction with the IFNγ promoter-protoxin transgene as described in Aim 1.

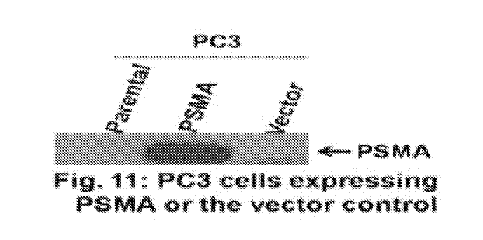

Sub-Aim 2b: Evaluate the homing and efficacy of genetically-engineered anti-PSMA CAR-targeted T-cells expressing PSA-activated proaerolysin (PA) in preclinical models of human PCa. Confirmation of PSMA-dependent expression of PA from the genetically-engineered T-cells generated in Sub-aim 2a are performed using PC3 cells stably expressing either PSMA or the vector control (FIG. 11)(71). Because PC3 cells do not express PSA, supernatants derived from PC3:T-cell co-culture can be incubated with PSA (AbD Serotec) and analyzed by western blot to determine PSA-dependent cleavage of the inhibitory domain. Cytotoxicity assays are performed using LNCaP cells (PSMA+ and PSA+) cultured in the presence or absence of a PSA inhibitor, Ahx-FSQn(boro)Bpg (FIG. 9)(106). The assay are performed with decreasing effector:target ratios and quantified using a standard chromium (51Cr) release assay (34) measured on a liquid scintillation counter (Beckman LS6000TA). The increased potency of these genetically-engineered T-cells are evaluated by comparing these results to those achieved with T-cells only expressing the anti-PSMA CAR (no protoxin expression) and the parental T-cell population. T-cell expansion are quantified using a Nexcel cellometer following co-culture with irradiated (30 Gy) PSMA-expressing PC3 cells.

In vivo efficacy are demonstrated using PCa xenograft models. First, the homing efficiency of genetically-modified T-cells relative to wildtype controls are evaluated using protocols described in Sub-aim 1b. Additionally, comparison of the homing efficiency to PC3-PSMA xenografts relative to contralateral PC3 controls (3/group) can be used to determine whether CAR-antigen recognition increases tumor engraftment and toxicity in the absence of PSA-mediated protoxin activation. Next, anti-tumor efficacy are quantified using subcutaneous CWR-22Rv1, LNCaP, or VCaP PCa xenografts (10/group) in NOG mice following IV injection of genetically-engineered T-cells (1×106). Importantly, all of these lines express PSMA and enzymatically-active PSA. Tumor measurements are taken twice weekly using digital calipers over a ≥1 month period to calculate tumor volumes according to previously published methods (43). A therapeutic response are defined as >50% regression in tumor volume with acceptable host toxicity (i.e., <10% loss in body weight and no deaths). Should these criteria be met, efficacy is further evaluated in both orthotopic (CWR-22r or LNCaP) and intratibial (CWR-22r, LNCaP, or VCaP) models of PCa, which may more accurately reflect the microenvironment present in primary and metastatic clinical disease. Each of these subcutaneous, orthotopic, and intratibial xenograft models are currently operational within the lab and are used routinely. If an insufficient therapeutic response is observed, the strategies described in Sub-Aim 1b, such as multiple doses, promoter modifications/exchange and transgene ‘stacking’ can be used to enhance anti-tumor efficacy.

Significance and Patient Impact: Prostate cancer (PCa) is the most frequently diagnosed cancer in men, and there are currently no curative treatment options once the disease progresses to a castration-resistant metastatic state. Tumor heterogeneity and dose-limiting toxicities associated with current treatment paradigms require that highly innovative new therapeutic approaches be pursued if we hope to successfully cure men of advanced PCa. Utilizing the innate oncotropic properties of T-cells to deliver a PSA-activated protoxin to the tumor microenvironment within sites of PCa represents just such an innovative strategy. By combining T-cells ability to recognize vanishingly small levels of antigens, in this case using an anti-PSMA CAR, to drive the expression of a highly potent pore-forming cytotoxin, such as PA, ‘on-target, off-tumor’ effects can be minimized and a significant enhancement of T-cell cytotoxic potency can be achieved. This can be accomplished using genetic engineering strategies to transform autologous T-cells into biological ‘microfactories’ capable of secreting large quantities of PSA-activated PA into the extracellular fluid of the tumor following appropriate antigenic stimulation by PSMA-expressing cells. PSA-activated PA is currently in phase III registration trials as a local therapy for symptomatic BPH. CARs have recently emerged as powerful tools to generate tumor-specific cytotoxic T-cells that have been validated in clinical trials for the treatment of relapsed chemotherapy-refractory ALL patients. However, it's their integration into a single therapeutic strategy using genetic engineering techniques to overcome intrinsic limitations and maximize their clinical potential that represents the true innovation of this invention and provides the unique opportunity for rapid translation into meaningful patient outcomes.

### II. Definitions

Aerolysin: A channel-forming toxin produced as an inactive protoxin called proaerolysin (PA) (wild-type PA is shown in SEQ ID NOS: 1 and 2). The PA protein contains many discrete functionalities that include a binding domain (approximately amino acids 1-83 of SEQ ID NO: 2), a toxin domain (approximately amino acids 84-426 of SEQ ID NO: 2), and a C-terminal inhibitory peptide domain (approximately amino acids 427-470 of SEQ ID NO: 2) that contains a protease activation site (amino acids 427-432 of SEQ ID NO: 2).

The binding domain recognizes and binds to glycophosphatidylinositol (GPI) membrane anchors including those found in Thy-1 on T lymphocytes, the PIGA gene product found in erythrocyte membranes, and Prostate Stem Cell Antigen (PSCA). Most mammalian cells express GPI anchored proteins on their surfaces. The activation or proteolysis site within wildtype PA is a six amino acid sequence that is recognized as a proteolytic substrate by the furin family of proteases. Wild-type PA is activated upon hydrolysis of a C-terminal inhibitory segment by furin. Activated aerolysin binds to GPI-anchored proteins in the cell membrane and forms a heptamer that inserts into the membrane producing well-defined channels of ˜17 Å. Channel formation leads to rapid cell death via necrosis. Wild-type aerolysin is toxic to mammalian cells, including erythrocytes, for example at 1 nanomolar or less.

Antibody: Immunoglobulin molecules and immunologically active portions of immunoglobulin molecules, i.e., molecules that contain an antigen binding site which specifically binds (immunoreacts with) an antigen. A naturally occurring antibody (e.g., IgG) includes four polypeptide chains, two heavy (H) chains and two light (L) chains inter-connected by disulfide bonds. However, the antigen-binding function of an antibody can be performed by fragments of a naturally occurring antibody. Thus, these antigen-binding fragments are also intended to be designated by the term antibody. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment which consists of a VH domain; (v) an isolated complimentarily determining region (CDR); and (vi) an F(ab′)2 fragment, a bivalent fragment comprising two Fab fragments linked by a disulfide bridge at the hinge region. Furthermore, although the two domains of the Fv fragment are coded for by separate genes, a synthetic linker can be made that enables them to be made as a single protein chain (known as single chain Fv (scFv)) by recombinant methods. Such single chain antibodies are also included. In one embodiment, an antibody includes camelized antibodies.

In one example, antibody fragments are capable of crosslinking their target antigen, e.g., bivalent fragments such as F(ab′)2 fragments. Alternatively, an antibody fragment which does not itself crosslink its target antigen (e.g., a Fab fragment) can be used in conjunction with a secondary antibody which serves to crosslink the antibody fragment, thereby crosslinking the target antigen. Antibodies can be fragmented using conventional techniques and the fragments screened for utility in the same manner as described for whole antibodies. An antibody is further intended to include bispecific and chimeric molecules that specifically bind the target antigen.

Specifically binds: Binding that occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. As used herein, the terms “specific binding” or “specifically binding” when used in reference to the interaction of an antibody and a protein or peptide means that the interaction is dependent upon the presence of a particular structure (i.e., the epitope) on the protein.

Cancer: Malignant neoplasm that has undergone characteristic anaplasia with loss of differentiation, increase rate of growth, invasion of surrounding tissue, and is capable of metastasis.

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Chemical synthesis: An artificial means by which one can make a protein or peptide. A synthetic protein or peptide is one made by such artificial means.

Chemotherapy: In cancer treatment, chemotherapy refers to the administration of one or a combination of compounds to kill or slow the reproduction of rapidly multiplying cells. Chemotherapeutic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol and taxotere. Such agents can be co-administered with the disclosed variant PA fusion proteins to a subject. Alternatively or in addition, chemotherapeutic agents can be administered prior to and/or subsequent to administration of the disclosed variant PA fusion proteins to a subject. In one example, chemotherapeutic agents are co-administered with hormonal and radiation therapy, along with the disclosed variant PA fusion proteins, for treatment of a localized prostate carcinoma.

Conservative substitution: One or more amino acid substitutions (for example 1, 2, 3, 4, 5, 6, 7, 8, 9, 10 or more residues) for amino acid residues having similar biochemical properties. Typically, conservative substitutions have little to no impact on the activity of a resulting polypeptide. For example, ideally, a modified PA peptide including one or more conservative substitutions retains proaerolysin activity. A polypeptide can be produced to contain one or more conservative substitutions by manipulating the nucleotide sequence that encodes that polypeptide using, for example, standard procedures such as site-directed mutagenesis or PCR.

Substitutional variants are those in which at least one residue in the amino acid sequence has been removed and a different residue inserted in its place. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Permissive substitutions are non-conservative amino acid substitutions, but also do not significantly alter proaerolysin activity. An example is substitution of Cys for Ala at position 300 of SEQ ID NO: 2 or 4. Further information about conservative substitutions can be found in, among other locations in, Ben-Bassat et al., (J. Bacteria 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology. In one example, such variants can be readily selected for additional testing by performing an assay to determine if the variant retains variant PA fusion protein activity.

Deletion: The removal of a sequence of a nucleic acid, for example DNA, the regions on either side being joined together.

DNA: Deoxyribonucleic acid. DNA is a long chain polymer which comprises the genetic material of most living organisms (some viruses have genes comprising ribonucleic acid, RNA). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Enhance: To improve the quality, amount, or strength of something. In one embodiment, a therapy enhances the ability of a subject to reduce tumors, such as a prostate carcinoma, in the subject if the subject is more effective at fighting tumors. In another embodiment, a therapy enhances the ability of an agent to reduce tumors, such as a prostate carcinoma, in a subject if the agent is more effective at reducing tumors. Such enhancement can be measured using the methods disclosed herein, for example determining the decrease in tumor volume.

Functional Deletion: A mutation, partial or complete deletion, insertion, or other variation made to a gene sequence which renders that part of the gene sequence nonfunctional. For example, functional deletion of a PA binding domain results in a decrease in the ability of PA to bind to and concentrate in the cell membrane. This functional deletion can be reversed by inserting another functional binding domain into proaerolysin, such as a prostate-specific binding domain, for example, an LHRH peptide.

Examples of methods that can be used to functionally delete a proaerolysin binding domain, include, but are not limited to: deletion of about amino acids 1-83 of SEQ ID NO: 2 or fragments thereof, such as about amino acids 45-66 of SEQ ID NO: 2, or inserting one or more of the following mutations into a variant proaerolysin sequence W45A, I47E, M57A, Y61A, K66Q (amino acid numbers refer to SEQ ID NO: 2) (for example, see Mackenzie et al. J. Biol. Chem. 274: 22604-22609, 1999). In another example, functional deletion of a native PA furin cleavage site results in a decrease in the ability of PA to be cleaved and activated by furin, when compared to a wild-type PA molecule.

Immobilized: Bound to a surface, such as a solid surface. A solid surface can be polymeric, such as polystyrene or polypropylene. In one embodiment, the solid surface is in the form of a bead. In another embodiment, the surface includes a modified PA toxin, and in some examples further includes one or more prostate-specific binding ligands, such as LHRH peptide, PSMA antibody, and PSMA single chain antibody. Ideally, the modified PA toxin is liberated from the bead once the bead reaches the prostate cell target. Methods of immobilizing peptides on a solid surface can be found in WO 94/29436, and U.S. Pat. No. 5,858,358. Examples of how the molecules can be attached to the bead include, but are not limited to: HSA-PSA cleavage site/linker-PA-bead-prostate binding ligand; or prostate binding ligand-bead-HSA-cleavage linker-PA.

Isolated: An “isolated” biological component (such as a nucleic acid molecule or protein) has been substantially separated or purified away from other biological components in the cell of the organism in which the component naturally occurs (i.e., other chromosomal and extrachromosomal DNA and RNA). Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. An isolated cell is one which has been substantially separated or purified away from other biological components of the organism in which the cell naturally occurs.

Malignant: Cells that have the properties of anaplasia invasion and metastasis.

Mammal: This term includes both human and non-human mammals. Similarly, the terms “subject” and “patient” are interchangeable and include both human and veterinary subjects. Examples of mammals include, but are not limited to, humans, pigs, cows, goats, cats, dogs, rabbits and mice.

Neoplasm: Abnormal growth of cells.

Normal Cell: Non-tumor cell, non-malignant, uninfected cell.

Oligonucleotide: A linear polynucleotide sequence of up to about 200 nucleotide bases in length, for example a polynucleotide (such as DNA or RNA) which is at least about 6 nucleotides, for example at least 15, 50, 100 or 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Polynucleotide: A linear nucleic acid sequence of any length. Therefore, a polynucleotide includes molecules which are at least 5, 15, 50, 100, 200, 400, 500, 1000, 1100, or 1200 (oligonucleotides) and also nucleotides as long as a full-length cDNA or chromosome.

Proaerolysin: The inactive protoxin of aerolysin. The cDNA and protein of a wild-type or native proaerolysin (PA) are shown in SEQ ID NOS: 1 and 2, respectively. In one example, a variant or modified proaerolysin molecule includes a prostate-specific protease cleavage site, such as a PSA-specific cleavage site, which permits activation of the variant PA in the presence of a prostate-specific protease such as PSA, PMSA, or HK2. In one example, a prostate-specific protease cleavage site is inserted into the native furin cleavage site of PA, such that PA is activated in the presence of a prostate-specific protease, but not furin. Alternatively, the furin cleavage site can be functionally deleted using mutagenesis of the six amino acid sequence, and a prostate-specific protease cleavage sequence can be inserted. In another example, a variant PA molecule further includes deletion or substitution of one or more of the native PA amino acids. In yet another example, a variant PA molecule further includes another molecule (such as an antibody or peptide) linked or added to (or within) the variant PA molecule. In another example, a variant PA molecule includes a prostate-tissue specific binding domain.

In another example, a variant PA molecule further includes a functionally deleted binding domain (about amino acids 1-83 of SEQ ID NO: 2). Functional deletions can be made using any method known in the art, such as deletions, insertions, mutations, or substitutions. Examples include, but are not limited to deleting the entire binding domain (or portions thereof) or introduction of point mutations, which result in a binding domain with decreased function. For example, a PA molecule which has a functionally deleted binding domain (and no binding sequence substituted therefor), will have a decreased ability to accumulate in a cell membrane, and therefore lyse cells at a slower rate than a wild-type PA sequence. Also disclosed are variant PA proteins in which the native binding domain is functionally deleted and replaced with a prostate-tissue specific binding domain as described below.

In another example, a variant or modified PA molecule includes a PSA cleavage site, and a functionally deleted binding domain which is replaced with a prostate-tissue specific binding domain. Such variant PA fusion proteins are targeted to prostate cells via the prostate-tissue specific binding domain, and activated in the presence of PSA.

Particular non-limiting examples of variant PSA proteins are shown in SEQ ID NOS: 4, 7, 10, 13, 24, and 25.

Modified PA activity is the activity of an agent in which the lysis of cells is affected. Cells include, but are not limited to prostate-specific protease secreting cells, such as PSA-secreting cells, such as prostate cancer cells, such as slow-proliferating prostate cancer cells. Agents include, but are not limited to, modified PA proteins, nucleic acids, specific binding agents, including variants, mutants, polymorphisms, fusions, and fragments thereof, disclosed herein. In one example, modified PA activity is said to be enhanced when modified PA proteins or nucleic acids, when contacted with a PSA-secreting cell (such as a prostate cancer cell), promote lysis and death of the cell, for example by at least 10%, or for example by at least 25%, 50%, 100%, 200% or even 500%, when compared to lysis of a non-PSA producing cell. In other examples, modified PA activity is said to be enhanced when modified PA proteins and nucleic acids, when contacted with a tumor, decrease tumor cell volume, such as a prostate tumor, for example by at least 10% for example by at least 20%, 30%, 40%, 50%, 60%, 70%, 75%, 80%, 85%, 90%, 95%, 98%, or even 100% (complete elimination of the tumor). Assays which can be used to determine if an agent has modified PA activity are described, for example, in U.S. Pat. No. 7,838,266, No. 7,745,395, and No. 7,282,476, which are all incorporated herein by reference.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Prostate-specific promoter: A promoter responsive to testosterone and other androgens, which therefore promotes gene expression in prostate cells. Examples include, but are not limited to the probasin promoter; the prostate specific antigen (PSA) promoter; the prostate specific membrane antigen (PSMA) promoter; and the human glandular kallikrein 2 (HK2) promoter.

Prostate-specific protease cleavage site: A sequence of amino acids which is recognized and specifically and efficiently hydrolyzed (cleaved) by a prostate-specific protease. Examples include, but are not limited to a PSA-specific cleavage site, a PSMA-specific cleavage site and an HK2-specific cleavage site. Variant PA fusion proteins of the present invention can comprise one or more cleavage sites/linkers. For example, albumin can be fused to the N-terminus of a variant PA protein using one, two, three, four, five, six or more prostate-specific protease cleavage site linkers.

PSA-specific cleavage site: is a sequence of amino acids which is recognized and specifically and efficiently hydrolyzed (cleaved) by prostate specific antigen (PSA). Such peptide sequences can be introduced into other molecules, such as PA, to produce prodrugs that are activated by PSA. Upon activation of the modified PA by PSA, PA is activated and can exert its cytotoxicity. Examples of PSA-specific cleavage sites include, but are not limited to, those shown in SEQ ID NOS: 5, 8 and 14-21, those disclosed in U.S. Pat. No. 6,391,305; No. 6,368,598; No. 6,265,540; No. 5,998,362; No. 5,948,750; and No. 5,866,679.

PSMA-specific cleavage site: Particular examples of PSMA-specific cleavage sites can be found in WO/0243773 to Isaacs and Denmeade (herein incorporated by reference). The PSMA cleavage site includes at least the dipeptide, X₁X₂. This peptide contains the amino acids Glu or Asp at position X₁. X₂ can be Glu, Asp, Gln, or Asn. Tripeptides X₁X₂X₃ are also suitable, with X₁ and X₂ defined as before, with X₃ as Glu, Asp, Gln or Asn. Tetrapeptides X₁X₂X₃X₄ are also suitable, with X₁-3 defined as above, and with X₄ as Glu, Asp, Gln or Asn. Pentapeptides X₁X₂X₃X₄X₅ are also suitable, with X₁-4 defined as above, and with X₅ as Glu, Asp, Gln or Asn. Hexapeptides X₁X₂X₃X₄X₅X₆ are also suitable, with X₁-5 defined as above, and with X₆ as Glu, Asp, Gln or Asn. Further peptides of longer sequence length can be constructed in similar fashion.

Generally, the peptides are of the following sequence: X₁ . . . Xₙ, where n is 2 to 30, preferably 2 to 20, more preferably 2 to 15, and even more preferably 2 to 6, where X₁ is Glu, Asp, Gln or Asn, but is preferably Glu or Asp, and X₂-Xₙ are independently selected from Glu, Asp, Gln and Asn. Some preferred peptide sequences are as above, except that X₂-Xₙ₋₁ are independently selected from Glu, and Asp, and Xₙ is independently selected from Glu, Asp, Gln and Asn. The length of the peptide can be optimized to allow for efficient PSMA hydrolysis, enhanced solubility of therapeutic drug in aqueous solution, if this is needed, and limited non-specific cytotoxicity in vitro.

HK2-specific cleavage site: Particular examples of HK2-specific cleavage sites are disclosed in WO01/09165 and U.S. Patent Publication No. 20120309692 and include, but are not limited to, Lys-Arg-Arg, Ser-Arg-Arg, Ala-Arg-Arg, His-Arg-Arg, Gln-Arg-Arg, Ala-Phe-Arg, Ala-Gln-Arg, Ala-Lys-Arg, Ala-Arg-Lys, Ala-His-Arg, Gln-Lys-Arg-Arg (SEQ ID NO:28), Lys-Ser-Arg-Arg (SEQ ID NO:29), Ala-Lys-Arg-Arg (SEQ ID NO:30), Lys-Lys-Arg-Arg (SEQ ID NO:31), His-Lys-Arg-Arg (SEQ ID NO:32), Lys-Ala-Phe-Arg (SEQ ID NO:33), Lys-Ala-Gln-Arg (SEQ ID NO:34), Lys-Ala-Lys-Arg (SEQ ID NO:35), Lys-Ala-Arg-Lys (SEQ ID NO:36), Lys-Ala-His-Arg (SEQ ID NO:37), His-Ala-Gln-Lys-Arg-Arg (SEQ ID NO:38), Gly-Gly-Lys-Ser-Arg-Arg (SEQ ID NO:39), His-Glu-Gln-Lys-Arg-Arg (SEQ ID NO:40), His-Glu-Ala-Lys-Arg-Arg (SEQ ID NO:41), Gly-Gly-Gln-Lys-Arg-Arg (SEQ ID NO:42), His-Glu-Gln-Lys-Arg-Arg (SEQ ID NO:43), Gly-Gly-Ala-Lys-Arg-Arg (SEQ ID NO:44), His-Glu-Gln-Lys-Arg-Arg (SEQ ID NO:45), Gly-Gly-Lys-Lys-Arg-Arg (SEQ ID NO:46), and Gly-Gly-His-Lys-Arg-Arg (SEQ ID NO:47).

PRX302: A modified proaerolysin where the furin site of proaerolysin has been replaced with a PSA-specific cleavage site. SEQ ID NOS: 3 and 4 show the PRX302 cDNA and protein sequence, respectively. SEQ ID NO:26 shows the protein sequence of SEQ ID NO: 4 with an N-terminal His tag. The term “PRX302” includes the proteins of both SEQ ID NO: 4 and SEQ ID NO:26.

Prostate tissue-specific binding domain: A molecule, such as a peptide ligand, toxin, or antibody, which has a higher specificity for prostate cells than for other cell types. In one example, a prostate tissue specific binding domain has a lower K_{D} in prostate tissue or cells than in other cell types, (i.e., binds selectively to prostate tissues as compared to other normal tissues of the subject), for example at least a 10-fold lower K_{D}, such as an at least 20-, 50-, 75-, 100- or even 200-fold lower K_{D}. Such sequences can be used to target an agent, such as a variant PA molecule, to the prostate. Examples include, but are not limited to: antibodies which recognize proteins that are relatively prostate-specific such as PSA, PSMA, hK2, prostasin, and hepsin; ligands which have prostate-selective receptors such as natural and synthetic luteinizing hormone releasing hormone (LHRH); and endothelin (binding to cognate endothelin receptor).

Purified: The term “purified” does not require absolute purity; rather, it is intended as a relative term. Thus, for example, a substantially purified protein or nucleic acid preparation (such as the modified PA toxins disclosed herein) is one in which the protein or nucleic acid referred to is more pure than the protein in its natural environment within a cell or within a production reaction chamber (as appropriate). For example, a preparation of a modified PA protein is purified if the protein represents at least 50%, for example at least 70%, of the total protein content of the preparation. Methods for purification of proteins and nucleic acids are well known in the art. Examples of methods that can be used to purify a protein, such as a modified PA, include, but are not limited to the methods disclosed in Sambrook et al. (Molecular Cloning: A Laboratory Manual, Cold Spring Harbor, N.Y., 1989, Ch. 17).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. A recombinant protein is one that results from expressing a recombinant nucleic acid encoding the protein.

Sample: Biological samples containing genomic DNA, cDNA, RNA, or protein obtained from the cells of a subject, such as those present in peripheral blood, urine, saliva, semen, tissue biopsy, surgical specimen, fine needle aspirates, amniocentesis samples and autopsy material. In one example, a sample includes prostate cancer cells obtained from a subject.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, 75%, 80%, 85%, 86%, 87%, 88%, 89%, 90%, 91%, 92%, 93%, 94%, 95%, 96%, 97%, 98 or even 99% sequence identity. When less than the entire sequence is being compared for sequence identity, homologs will typically possess at least 75% sequence identity over short windows of 10-20 amino acids, and can possess sequence identities of at least 85%, 86%, 87%, 88%, 89%, 90%, 91%, 92%, 93%, 94%, 95%, 96%, 97%, 98% or even 99% depending on their identity to the reference sequence. Methods for determining sequence identity over such short windows are described at the NCBI web site.

Protein homologs are typically characterized by possession of at least 70%, such as at least 75%, 80%, 85%, 86%, 87%, 88%, 89%, 90%, 91%, 92%, 93%, 94%, 95%, 96%, 97%, 98% or even 99% sequence identity, counted over the full-length alignment with the amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG.

One of skill in the art will appreciate that these sequence identity ranges are provided for guidance only; it is possible that strongly significant homologs could be obtained that fall outside the ranges provided. Provided herein are the peptide homologs described above, as well as nucleic acid molecules that encode such homologs.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous peptides can, for example, possess at least 75%, 80%, 85%, 86%, 87%, 88%, 89%, 90%, 91%, 92%, 93%, 94%, 95%, 96%, 97%, 98% or even 99% sequence identity determined by this method. When less than the entire sequence is being compared for sequence identity, homologs can, for example, possess at least 75%, 85%, 86%, 87%, 88%, 89%, 90%, 91%, 92%, 93%, 94%, 95%, 96%, 97%, 98% or even 99% sequence identity over short windows of 10-20 amino acids. Methods for determining sequence identity over such short windows can be found at the NCBI web site. One of skill in the art will appreciate that these sequence identity ranges are provided for guidance only; it is possible that significant homologs or other variants can be obtained that fall outside the ranges provided.

Subject: Living multicellular vertebrate organisms, a category which includes both human and veterinary subjects that require an increase in the desired biological effect. Examples include, but are not limited to: humans, apes, dogs, cats, mice, rats, rabbits, horses, pigs, and cows. The term “subject” can be used interchangeably with the term “patient.”

Therapeutically Effective Amount: An amount sufficient to achieve a desired biological effect, for example, an amount that is effective to decrease the size (i.e., volume), side effects and/or metastasis of prostate cancer. In one example, it is an amount sufficient to decrease the symptoms or effects of a prostate carcinoma, such as the size of the tumor. In particular examples, it is an amount effective to decrease the size of a prostate tumor and/or prostate metastasis by at least 30%, 40%, 50%, 70%, 80%, 85%, 86%, 87%, 88%, 89%, 90%, 91%, 92%, 93%, 94%, 95%, 96%, 97%, 98%, 99% or even 100% (complete elimination of the tumor).

In particular examples, it is an amount of a variant PA fusion protein effective to decrease a prostate tumor and/or an amount of prostate cancer cells lysed by a variant PA fusion protein, such as in a subject to whom it is administered, for example a subject having one or more prostate carcinomas. In other examples, it is an amount of a variant PA fusion protein and/or an amount of prostate cancer cells lysed by such a variant PA fusion protein, effective to decrease the metastasis of a prostate carcinoma.

In one embodiment, the therapeutically effective amount also includes a quantity of a variant PA fusion protein and/or an amount of prostate cancer cells lysed by a variant PA fusion protein sufficient to achieve a desired effect in a subject being treated. For instance, these can be an amount necessary to improve signs and/or symptoms a disease such as cancer, for example prostate cancer.

An effective amount of a variant PA fusion protein and/or prostate cancer cells lysed by such a variant PA fusion protein can be administered in a single dose, or in several doses, for example daily, during a course of treatment. However, the effective amount of are dependent on the subject being treated, the severity and type of the condition being treated, and the manner of administration. For example, a therapeutically effective amount of a variant PA fusion protein can vary from about 1-10 mg per 70 kg body weight, for example about 2.8 mg, if administered iv and about 10-100 mg per 70 kg body weight, for example about 28 mg, if administered intraprostatically or intratumorally. In addition, a therapeutically effective amount of prostate cancer cells lysed by PA (variant or wild-type) can vary from about 10⁶ to 10⁸ cells.

Therapeutically effective dose: In one example, a dose of a variant PA fusion protein sufficient to decrease tumor cell volume, such as a prostate carcinoma, in a subject to whom it is administered, resulting in a regression of a pathological condition, or which is capable of relieving signs or symptoms caused by the condition. In a particular example, it is a dose of a variant PA fusion protein sufficient to decrease metastasis of a prostate cancer.

In yet another example, it is a dose of cell lysate resulting from contact of cells with a variant PA fusion protein sufficient to decrease tumor cell volume, such as a prostate carcinoma, in a subject to whom it is administered, resulting in a regression of a pathological condition, or which is capable of relieving signs or symptoms caused by the condition. In a particular example, it is a dose of cell lysate resulting from contact of cells with a modified or wild-type PA sufficient to decrease metastasis of a prostate cancer.

Tumor: A neoplasm. Includes solid and hematological (or liquid) tumors. Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenström's macroglobulinemia, heavy chain disease, myelodysplastic syndrome, mantle cell lymphoma and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Transformed: A transformed cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: Transformed cells which contain foreign, non-native DNA.

Transgenic mammal: Transformed mammals which contain foreign, non-native DNA. In one embodiment, the non-native DNA is a modified PA which includes HSA fused to the N-terminus of PA using a prostate-specific protease cleavage site.

Variants or fragments or fusion proteins: The production of a variant PA fusion protein can be accomplished in a variety of ways (for example see Examples 12 and 16 of U.S. Pat. No. 7,838,266, No. 7,745,395, and No. 7,282,476, which are all incorporated herein by reference). DNA sequences which encode for a variant PA fusion protein, or a fragment or variant of a variant PA fusion protein (for example a fragment or variant having 80%, 85%, 86%, 87%, 88%, 89%, 90%, 91%, 92%, 93%, 94%, 95%, 96%, 97%, 98% or even 99% sequence identity to a variant PA fusion protein) can be engineered to allow the protein to be expressed in eukaryotic cells or organisms, bacteria, insects, and/or plants. To obtain expression, the DNA sequence can be altered and operably linked to other regulatory sequences. The final product, which contains the regulatory sequences and the therapeutic variant PA fusion protein, is referred to as a vector. This vector can be introduced into eukaryotic, bacteria, insect, and/or plant cells. Once inside the cell the vector allows the protein to be produced.

A fusion protein which includes a modified PA, (or variants, polymorphisms, mutants, or fragments thereof) linked to other amino acid sequences that do not inhibit the desired activity of the protein, for example the ability to lyse tumor cells. In one example, the other amino acid sequences are no more than 5, 6, 7, 8, 9, 10, 20, 30, or 50 amino acid residues in length. In other embodiments, a modified PA is fused to another peptide/protein that is more than 50 amino acids in length including, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600 or more.

One of ordinary skill in the art will appreciate that the DNA can be altered in numerous ways without affecting the biological activity of the encoded protein. For example, PCR can be used to produce variations in the DNA sequence which encodes a variant PA toxin. Such variants can be variants optimized for codon preference in a host cell used to express the protein, or other sequence changes that facilitate expression.

Vector: A nucleic acid molecule as introduced into a host cell, thereby producing a transformed host cell. A vector can include nucleic acid sequences that permit it to replicate in the host cell, such as an origin of replication. A vector can also include one or more selectable marker genes and other genetic elements known in the art.

### III. Variant Proaerolysin Molecules

Bacterial toxins, such as aerolysin produced by Aeromonas hydrophilia and α-hemolysin produced by Staph aureus, are beta-sheet proteins that oligomerize in the plasma membrane to produce pores that lead to rapid cytolytic cell death. Pore formation physically disrupts the cell membranes, and results in death of cells in all phases of the cell cycle, including non-proliferating cells (i.e., G0 arrested). However, wild-type aerolysin kills cells indiscriminately. Herein disclosed is a fusion protein comprising human serum albumin and the inactive protoxin form of aerolysin that is activated by cleavage of the activation domain with a prostate-specific protease that also cleaves the HSA bulk protein (a variant PA) that can be targeted to, and activated by, prostate cancer specific proteins. One advantage of the disclosed variant PA fusion proteins for treatment of localized and metastatic prostate cancer is that it combines a proliferation independent therapy with prostate-specific drug delivery, resulting in minimal side effects to patients. One skilled in the art will understand that other protoxins, such as Clostridium septicum alpha toxin, Bacillus thuringiensis delta-toxin, and human perforin, bouganin, Pseudomonas exotoxin, Bcl-2, Cholera toxin, Abrin, Ricin, Verotoxin, Diptheria toxin, Tetanus toxin, Botulinum toxin, Neural thread protein, and Ribnuclease A can be substituted for proaerolysin.

Disclosed herein are variant PA fusion proteins, including both DNA and protein sequences, which include a prostate-specific protease cleavage sequence. Such variants are also fused with albumin using at least one prostate-specific protease cleavage sequence/linker (including one, two, three, four, five or more consecutive linkers). Examples of prostate-specific protease cleavage sequences include, but are not limited to: PSA, PSMA, and HK2 cleavage sequences. The prostate-specific protease cleavage sequence functionally replaces the native furin cleavage site of wild-type PA. This replacement results in a proaerolysin variant that only becomes cytolytically active in the presence of enzymatically active proteases such as PSA, PSMA, or hK2. PSA is a serine protease with the ability to recognize and hydrolyze specific peptide sequences. It is secreted by normal and malignant prostate cells in an enzymatically active form and becomes inactivated upon entering the circulation. Since neither blood nor normal tissue other than the prostate contains enzymatically active PSA, the proteolytic activity of PSA was used to activate protoxins at sites of prostate cancer. Any PSA, PSMA, or hK2 cleavage site can be used. Examples of PSA cleavage sites include, but are not limited to, those shown in SEQ ID NOS: 5, 8, 11, and 14-21. In a particular example, the PSA cleavage site includes SEQ ID NO: 5.

In some examples, the furin cleavage site of PA (amino acids 427-432 of SEQ ID NO: 2) is deleted and a prostate-specific protease cleavage site, such as a PSA cleavage site, is inserted. In other examples, the furin cleavage site of PA is mutated and a prostate-specific protease cleavage site, such as a PSA cleavage site, inserted within, or added to the N- or C-terminus of the furin site.

Also disclosed are variant PA fusion proteins in which the PA binding domain is functionally deleted. Such variant PA fusion proteins can contain a native furin cleavage site, whereby targeting to prostate cells is achieved by functionally replacing the PA binding domain with a prostate-tissue specific binding domain. Alternatively, variant PA fusion proteins contain a prostate-specific protease cleavage site, whereby activation of the protoxin primarily occurs in cells that secrete a prostate-specific protease. The PA binding domain includes about amino acids 1-83 of SEQ ID NO: 2. The binding domain can be functionally deleted using any method known in the art, for example by deletion of all or some of the amino acids of the binding domain, such as deletion of amino acids 1-83 of SEQ ID NO: 2 or 4, or such as deletion of one or more amino acids shown as amino acids 45-66 of SEQ ID NO: 2 or 4. In other examples, the binding domain is functionally deleted by introduction of one or more site-specific mutations into the variant PA sequence, such as W45A, I47E, M57A, Y61A, and K66Q of SEQ ID NO: 2 or 4.

Variant PA fusion proteins which include a prostate-tissue specific binding domain which functionally substitutes for the native PA binding domain are disclosed. The use of one or more prostate-tissue specific binding domains can increase targeting of the disclosed variant PA fusion proteins to the prostate cells and its metastases. Several prostate-tissue specific binding domains are known. Examples include, but are not limited to a luteinizing hormone releasing hormone (LHRH) sequence, such as those shown in SEQ ID NOS: 22 and 23, and antibodies that recognize PSA and/or PSMA.

One or more prostate-tissue specific binding domains can be linked to one or more amino acids of the disclosed variant PA fusion proteins, but ideally, do not interfere significantly with the ability of the variant PA to be activated by a prostate-specific protease such as PSA, and the ability to form pores in cell membranes. For example, prostate tissue specific binding domains can be linked or inserted at an N- and/or C-terminus of a variant PA In some examples, the native binding domain of PA is deleted (i.e., amino acids 1-83 of SEQ ID NO: 2 or 4), such that attachment or linking of a prostate tissue specific binding domain to the N-terminus results in attachment to amino acid 84 of SEQ ID NO: 2 or 4. In other examples, smaller deletions or point mutations are introduced into the native binding domain of PA, such that attachment or linking of a prostate tissue specific binding domain to the N-terminus results in attachment to amino acid 1 of SEQ ID NO: 2 or 4 (or whichever amino acid is N terminal following functional deletion of the native PA binding domain). In some examples, the N-terminal amino acid of PA is changed to a Cys or other amino acid to before attaching a prostate-tissue specific binding domain, to assist in linking the prostate-tissue specific binding domain to the variant PA protein.

Alternatively or in addition, one or more prostate tissue specific binding domains can be attached or linked to other amino acids of a variant PA molecule, such as amino acid 215 or 300 of SEQ ID NO: 2 or 4. In some examples, a Cys amino acid replaces the native amino acid at that position. For example, the following changes can be made to SEQ ID NO: 2 or 4: Tyr215Cys or Ala300Cys. In one example, where the prostate tissue specific binding domain is an antibody, crosslinking can be used to attach antibodies to a variant PA, for example by reacting amino groups on the antibody with cysteine located in the PA variant (such as amino acids Cys19, Cys75, Cys159, and/or Cys164 of SEQ ID NO: 2).

Also disclosed are particular variant PA fusion proteins, such as those shown in SEQ ID NOS: 3, 4, 6, 7, 9, 10, 12, 13, 24 and 25.

In some examples the disclosed variant PA fusion proteins are linked or immobilized to a surface, such as a bead. The bead can also include a prostate-specific ligand to enhance targeting to a prostate cell, such as a localized or metastasized prostate cancer cell.

In specific embodiments, a T-cell can be engineered to express a prodrug composition. In one embodiment, a prodrug composition comprises a prostate-specific antigen (PSA)-activated pro-aerolysin (PA), wherein a PSA cleavable linker replaces the native furin cleavage site within PA; and human serum albumin (HSA) or a fragment thereof fused to the N-terminus of the PSA-activated PA. In one embodiment, the PSA cleavable linker comprises SEQ ID NO:5. In another embodiment, the PSA cleavable linker replaces the amino acids at position 427-432 of SEQ ID NO:2. In yet another embodiment, the HSA or fragment thereof comprises the C-terminal end of HSA. In a specific embodiment, the HSA or fragment thereof comprises SEQ ID NO:27. In yet another specific embodiment, the HSA or fragment thereof is fused to the N-terminus of the PSA-activated PA with at least one PSA-cleavable linker sequence. In a particular embodiment, the at least one PSA-cleavable linker sequence comprises SEQ ID NO:5. The at least one PSA-cleavable linker sequence can be a series of identical linker sequences or a combination of sequences and includes at least 1, 2, 3, 4, 5, 6, 7, 8, 9, 10 or more linker sequences. In a more particular embodiment, the HSA or fragment thereof is fused to the N-terminus of the PSA-activated PA with four identical PSA-cleavable linker sequences, wherein the linker sequence comprises SEQ ID NO:5.

In particular embodiments, a T-cell can be engineered to express a recombinant protein comprising SEQ ID NO:48. In a specific embodiment, the protein further comprises a polyhistidine tag. In a more specific embodiment, the polyhistidine tag comprises six histidines at the C-terminus of SEQ ID NO:48.

In further embodiments, a T-cell can be engineered to express a prodrug composition comprising a prostate-specific protease-activated pro-aerolysin (PA), wherein a prostate-specific protease cleavable linker replaces the native furin cleavage site within PA; and a blood plasma protein or a fragment thereof fused to the N-terminus of the PSA-activated PA. In one embodiment, the prostate-specific protease comprises PSA, prostate specific membrane antigen (PSMA), or human glandular kallikrein 2 (HK2). In certain embodiments, the blood plasma protein comprises albumin. In a more specific embodiment, the blood plasma protein comprises human serum albumin.

### IV Human Serum Albumin

The T-cells of the present invention can also be engineered to express a bulky protein fused to a PA protein described herein. The contents of U.S. Provisional Patent Application No. 61/104,275, are hereby incorporated by reference.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, “albumin” refers collectively to albumin protein or amino acid sequence, or an albumin fragment or variant, having one or more functional activities (e.g., biological activities) of albumin. In particular, “albumin” refers to human albumin or fragments thereof (see EP 201 239, EP 322 094 WO 97/24445, WO95/23857) or albumin from other vertebrates or fragments thereof, or analogs or variants of these molecules or fragments thereof.

As used herein, the albumin portion of the fusion protein may comprise the full length of the sequence as shown in SEQ ID NO:27, or may include one or more fragments thereof that are capable preventing, substantially reducing or reducing binding of the recombinant PRX302 pro-drug protein to GPI-anchored proteins on normal cells in the blood or host tissues. In one embodiment, the HA protein fragment comprises the N-terminal end of HA. In another embodiment, the HA protein fragment comprises the C-terminal end of HA. In particular embodiments, HA fragments may comprise 10 or more amino acids in length or may comprise about 15, 20, 25, 30, 50, or more contiguous amino acids from the HA sequence or may include part or all of specific domains of HA. For instance, one or more fragments of HA spanning the first two immunoglobulin-like domains may be used.

The albumin portion of the albumin fusion proteins of the invention may be a variant of normal HA. The term “variants” includes insertions, deletions and substitutions, either conservative or non-conservative, where such changes do not substantially alter one or more of the oncotic, useful ligand-binding and non-immunogenic properties of albumin.

In particular, the albumin fusion proteins of the invention may include naturally occurring polymorphic variants of human albumin and fragments of human albumin, for example those fragments disclosed in EP 322 094 (namely HA (Pn), where n is 369 to 419). The albumin may be derived from any vertebrate, especially any mammal, for example human, cow, sheep, or pig. Non-mammalian albumins include, but are not limited to, hen and salmon. The albumin portion of the fusion protein may be from a different animal than the PRC302 portion.

Generally speaking, an HA fragment or variant are at least 100 amino acids long, preferably at least 150 amino acids long. The HA variant may consist of or alternatively comprise at least one whole domain of HA, for example domains 1 (amino acids 1-194 of SEQ ID NO:27), 2 (amino acids 195-387 of SEQ ID NO:27), 3 (amino acids 388-585 of SEQ ID NO:27), 1+2 (1-387 of SEQ ID NO:27), 2+3 (195-585 of SEQ ID NO:27 (amino acids 1-194 of SEQ ID NO:27+amino acids 388-585 of SEQ ID NO:27). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

In certain embodiments, the albumin portion of an albumin fusion protein of the invention comprises at least one subdomain or domain of HA or conservative modifications thereof.

The present invention relates generally to fusion proteins comprising albumin and methods of treating, preventing, or ameliorating diseases or disorders. A fusion protein comprising albumin refers to a protein formed by the fusion of at least one molecule of albumin (or a fragment or variant thereof) to at least one molecule of a PRX302 protein (or fragment or variant thereof). An albumin-PA or albumin-PRX302 fusion protein comprises at least a fragment or variant of a PA protein and at least a fragment or variant of human serum albumin, which are associated with one another, preferably by genetic fusion (i.e., the albumin fusion protein is generated by translation of a nucleic acid in which a polynucleotide encoding all or a portion of a PA/PRX302 protein is joined in-frame with a polynucleotide encoding all or a portion of albumin) or chemical conjugation to one another. The PA/PRX302 protein and albumin protein, once part of the fusion protein, may be referred to as a “portion”, “region” or “moiety” of the fusion protein.

In one embodiment, the invention provides a fusion protein comprising, or alternatively consisting of, a PA/PRX302 protein and a serum albumin protein. In other embodiments, the invention provides a fusion protein comprising, or alternatively consisting of, a biologically active and/or therapeutically active fragment of a PA/PRX302 protein and a serum albumin protein. In other embodiments, the invention provides a fusion protein comprising, or alternatively consisting of, a biologically active and/or therapeutically active variant of a PA/PRX302 protein and a serum albumin protein. In particular embodiments, the serum albumin protein component of the fusion protein is the mature portion of serum albumin.

In further embodiments, the invention provides a fusion protein comprising, or alternatively consisting of, a PA/PRX302 protein, and a biologically active and/or therapeutically active fragment of serum albumin. In further embodiments, the invention provides a fusion protein comprising, or alternatively consisting of, a PA/PRX302 protein and a biologically active and/or therapeutically active variant of serum albumin. In certain embodiments, the PA/PRX302 protein portion of the fusion protein is the full length of the PA/PRX302 protein.

In further embodiments, the invention provides a fusion protein comprising or alternatively consisting of, a biologically active and/or therapeutically active fragment or variant of a PA/PRX302 protein and a biologically active and/or therapeutically active fragment or variant of serum albumin. In some embodiments, the invention provides a fusion protein comprising, or alternatively consisting of, the mature portion of a PA/PRX302 protein and the mature portion of serum albumin.

In specific embodiments, the fusion protein comprises HA as the N-terminal portion, and a PA/PRX302 protein as the C-terminal portion. Alternatively, a fusion protein comprising HA as the C-terminal portion, and a PA/PRX302 protein as the N-terminal portion may also be used.

In other embodiments, the fusion protein has a PA/PRX302 protein fused to both the N-terminus and the C-terminus of albumin. In one embodiment, the PA/PRX302 proteins fused at the N- and C-termini are the same PA/PRX302 proteins. In other embodiments, the PA/PRX302 proteins fused at the N- and C-termini are different PA/PRX302 proteins or just different proteins. In another embodiment, the PA/PRX302 proteins fused at the N- and C-termini are different therapeutic proteins which may be used to treat or prevent the same disease, disorder, or condition.

In addition to the fusion protein in which the albumin portion is fused N-terminal and/or C-terminal of the PA/PRX302 protein portion, fusion proteins of the invention may also be produced by inserting the PA/PRX302 protein or peptide of interest into an internal region of HA. For instance, within the protein sequence of the HA molecule a number of loops or turns exist between the end and beginning of α-helices, which are stabilized by disulphide bonds. The loops, as determined from the crystal structure of HA (PDB identifiers 1AO6, 1BJ5, 1BKE, 1BM0, 1E7E to 1E71 and 1UOR) for the most part extend away from the body of the molecule. These loops are useful for the insertion, or internal fusion, of therapeutically active peptides, particularly those requiring a secondary structure to be functional, or PA/PRX302 proteins, to essentially generate an albumin molecule with specific biological activity.

Loops in human albumin structure into which peptides or polypeptides may be inserted to generate albumin fusion proteins of the invention include: Val54-Asn61, Thr76-Asp89, Ala92-Glu100, Gln170-Ala176, His247-Glu252, Glu266-Glu277, Glu280-His288, Ala362-Glu368, Lys439-Pro447, Val462-Lys475, Thr478-Pro486, and Lys50G-Thr566. In specific embodiments, peptides or polypeptides are inserted into the Val54-Asn61, Gin170-Ala176, and/or Lys560-Thr566 loops of mature human albumin (SEQ ID NO:27).

1. A T-cell engineered to express a prostate-specific antigen (PSA)-activated pro-aerolysin (PA) upon tumor antigen recognition by a chimeric antigen receptor (CAR) expressed on the surface of the T-cell.

2. The T-cell of claim 1, wherein the T-cell expresses more than one type of tumor antigen recognizing CAR.

3. The T-cell of claim 1, wherein the tumor antigen comprises prostate specific membrane antigen (PSMA) and/or prostate stem cell antigen (PSCA).

4. A T-cell engineered (a) to express at least one CAR that binds tumor antigens; and (b) to inducibly express a prostate-specific antigen (PSA)-activated pro-aerolysin (PA) upon tumor antigen recognition by CAR.

5. A T-cell engineered to express a protoxin upon tumor antigen recognition by a CAR expressed on the surface of the T-cell.

6. The T-cell of claim 5, wherein the protoxin is activated via cleavage by a cancer specific protease.
